(19) United States
(12) Patent Application Publication
(10) Pub. No.: US 2018/0200572 A1
(43) Pub. Date: Jul. 19, 2018

(54) SPORTS STRAP

(71) Applicant: Astrid Christine Wagner, Vienna (AT)

(72) Inventor: Astrid Christine Wagner, Vienna (AT)

(21) Appl. No.: 15/408,715

(22) Filed: Jan. 18, 2017

(51) Int. Cl. A63B 23/02; A61F 5/01; A63B 21/04; A63B 21/055; A63B 21/00

(52) U.S. Cl. CPC A63B 23/0233; A61F 5/01; A63B 21/04; A63B 21/0557; A63B 23/0205; A63B 21/4013; A63B 21/4015; A63B 21/4025; A63B 21/4039; A63B 21/4009

(57) ABSTRACT

A sports strap that helps the wearer improve their posture. The device aligns the pelvis horizontally and vertically and strengthens and/or relaxes muscles of the abdomen, the lower back and the lower extremities. The strap may be worn in more than 20 different variations. Depending on the variation different parts of the body will be effected in different ways. There is a choice of activation or relaxation. Wearing the strap in active motion (including inflexible pelvis strap) makes walking harder by trying to mimic a stair stepping motion, which activates muscles of the lower extremities. It increases the energy expenditure required for walking and helps burn additional calories. Wearing the strap in relaxed motion (only elastic strap, without inflexible pelvis strap) makes the whole body let go of strain, especially the lower part of the body.

### BACKGROUND OF THE INVENTION

### Field of the Invention

The present invention relates generally to body mechanics and, more particularly, to a strap and methods of wearing a strap that help improve the user's/wearer's alignment and posture through the use of sensorimotor stimulation.

### Description of the Related Technology

The importance of good posture is well known among health professionals. Posture generally refers to the alignment of the body and, more specifically, to the relative static and dynamic positioning of the body and its limbs. Ideally, in a body exhibiting good posture, the spine has no lateral curvature and the legs have little angulation in the knees and ankles.

The effects of poor posture are well documented and include limited range of motion wherein muscles, such as the pectoral muscles in the chest, may be permanently shortened due to a hunched-over upper body position. Other effects of poor posture include discomfort in the form of headaches at the base of the skull and pain in the shoulders, arms, and hands. Additionally, poor posture may lead to pain in the jaw due to a forward-head position, as well as decreased lung capacity due to decreased volume of the chest cavity and lungs. One of the most common consequences of poor posture is the onset of lower back pain, which may increase with advancing age. Finally, a hunched-over upper body position of poor posture can add years to one's appearance.

However, by assuming good posture, gravitational forces may be more evenly distributed through the bones, ligaments and muscles of the body. Maintaining good posture is equally important during passive (static) activities such as sitting and standing, as well as during dynamic activities such as walking, running, and lifting. Theoretically, the slight S-shape of the spine that is characteristic of good posture should preferably be maintained during both static and dynamic activities. Unfortunately, our increasingly sedentary lifestyle, wherein many hours are spent sitting in front of a computer, driving an automobile, or watching television, has an adverse effect on posture. Such static activities may result in forward protrusion of the head and neck, rounding of the thoracic and lumbar spine, stretching of the spinal ligaments and also causing abnormal tilting and rotation of the pelvis. This also puts added stress on the hips, joints and other portions of the lower body. Such stretching gradually causes pain in the neck, upper back, lower back and hips and results in fatiguing of the body's muscles and ligaments. Poor posture during dynamic activities such as running and lifting results in inefficient body movements with increased stress on muscles and ligaments.

Maintaining an awareness of proper posture during static and dynamic activities may sometimes prevent overstressing of muscles and ligaments. In addition, maintaining an awareness of proper posture may train certain muscles through muscle memory such that proper posture eventually becomes a habit. However, it is sometimes difficult to maintain an awareness of proper posture during mentally challenging activities such as working at a computer, or during physically challenging activities such as participating in aggressive sports. Physical therapists may utilize postural therapy to improve the posture of a patient. Such postural therapy may include techniques such as shoulder taping and breathing exercises. Conventional chiropractic techniques may utilize body manipulation and treatment to help keep the patient's spine in alignment. However, the patient must take the time and energy to visit the offices of a physical therapist or a chiropractor in order to receive the needed postural therapy and/or chiropractic manipulation. Moreover, an alternative treatment's predictable therapeutic response may be achieved through cutaneous nerve stimulation, but is often overlooked as a method to improve the patient's posture.

Therefore, there exists a need in the art for a strap/band for improving posture that may be worn in many different variations. In addition, there exists a need in the art for a strap that focuses on the front and/or the backside of the body. Furthermore, there exists a need in the art for a strap focusing on alignment and exercise of one or more toes. There also exists a need in the art for a strap that comprises a mix of elastic and inelastic sections, in combination with hip-to-feet connection, in order to provide activity of lower extremities. There exists a need in the art for a strap with a compact design to ensure convenient wearability, especially under everyday clothes and/or during other sports activities. Finally, there exists a need in the art for a strap capable of adjustability in size and intensity.

### SUMMARY

### Brief Description of the Drawings

FIGS. 1a-e show various perspectives of a sports strap, in accordance with an embodiment of the present invention.

FIG. 1a is a front view of the sports strap as worn.

FIG. 1b is a front oblique view of the sports strap as worn.

FIG. 1c is a back view of the sports strap as worn.

FIG. 1d is a side view of the sports strap as worn.

FIG. 1e is a sectional view of the foot strap as worn.

FIGS. 2a-c show an overview of pre-defined rings & straps, in accordance with an embodiment of the present invention.

FIG. 2a shows the pelvis strap, leg straps, and foot straps.

FIG. 2b shows the straps spliced together.

FIG. 2c shows embodiment-related, pre-defined rings & straps.

FIGS. 3a-j show various embodiments of the sports strap.

FIG. 3a shows a sports strap, in accordance with an embodiment of the present invention.

FIG. 3b shows a sports strap, in accordance with an embodiment of the present invention.

FIG. 3c shows a sports strap, in accordance with an embodiment of the present invention.

FIG. 3d shows a sports strap, in accordance with an embodiment of the present invention, which comprises two center rings and a center strap.

FIG. 3e shows a sports strap, in accordance with an embodiment of the present invention, which comprises two center rings, a center strap, two elongation straps and two elongation rings.

FIG. 3f shows a sports strap, in accordance with an embodiment of the present invention, which comprises one center ring.

FIG. 3g shows a sports strap, in accordance with an embodiment of the present invention, which comprises one center ring, two elongation straps and two elongation rings.

FIG. 3h shows a sports strap, in accordance with an embodiment of the present invention, which comprises two center rings, one center strap and two long leg straps.

FIG. 3i shows a sports strap, in accordance with an embodiment of the present invention, which comprises two center rings and two ankle rings.

FIG. 3j shows a sports strap, in accordance with an embodiment of the present invention, which comprises two center rings, two long leg straps and two ankle rings.

FIGS. 4a-j show a sports strap, in accordance with an embodiment of the present invention fitted with comfort/adjustability-related options like length adjustments and/or open/close mechanisms.

FIG. 4a.1 shows a sports strap, in accordance with an embodiment of the present invention in which pelvis strap 20 is exemplary fitted with press buttons 40, and foot straps 22 are exemplary fitted with press buttons 40 as well.

FIG. 4a.2 shows a backside view of a sports strap, in accordance with an embodiment of the present invention, fitted with the comfort/adjustability related options as shown in FIG. 4a.1.

FIG. 4a.3 shows a foot strap 22 of a sports strap, in accordance with an embodiment of the present invention, fitted with the comfort/adjustability related options as shown in FIG. 4a.1.

FIG. 4b.1 shows a sports strap, in accordance with an embodiment of the present invention, in which pelvis strap 20 is exemplary fitted with buckles 43; center strap 310 is fitted with hook 41 & eye 42 fasteners; and foot straps 22 are fitted with hook 41 & eye 42 fasteners.

FIG. 4b.2 shows a front view of a sports strap, in accordance with an embodiment of the present invention, fitted with the comfort/adjustability related options as shown in FIG. 4b.1.

FIG. 4b.3 shows a foot strap 22 of a sports strap, in accordance with an embodiment of the present invention, fitted with comfort/adjustability related options as shown in FIG. 4b.1.

FIG. 4c.1 shows a sports strap, in accordance with an embodiment of the present invention, in which pelvis strap 20 is exemplary fitted with hook 41 & eye 42 fasteners; leg straps 21 are fitted with slides 44 and hook 41 & eye 42 fasteners; and foot straps 22 are fitted with press buttons 40.

FIG. 4c.2 shows a front view of a sports strap, in accordance with an embodiment of the present invention, fitted with the comfort/adjustability related options as shown in FIG. 4c.1.

FIG. 4d.1 shows a sports strap, in accordance with an embodiment of the present invention, in which pelvis strap 20 is fitted with press buttons 40; center strap 310 is fitted with press buttons 40; leg straps 21 are fitted with press buttons 40; and foot straps 22 are fitted with press buttons 40.

FIG. 4d.2 is a front view of a sports strap, in accordance with an embodiment of the present invention, fitted with the comfort/adjustability related options as shown in FIG. 4d.1.

FIG. 4e.1 shows a sports strap, in accordance with an embodiment of the present invention, in which pelvis strap 20, center strap 310, leg straps 21 and top end 341 of elongation straps 340 are fitted with press buttons; and foot straps 22 as well as bottom end 342 of elongation straps 340 and elongation rings 330 are held together with a stitched seam 45.

FIG. 4e.2 is a front view of a sports strap, in accordance with an embodiment of the present invention, fitted the comfort/adjustability related options as shown in FIG. 4e.1.

FIG. 4f.1 shows a sports strap, in accordance with an embodiment of the present invention, in which pelvis strap 20, leg straps 21 and foot straps 22 are fitted with hook 41 and eye 42 fasteners.

FIG. 4f.2 shows a front view of a sports strap, in accordance with an embodiment of the present invention, fitted with the comfort/adjustability related options as shown in FIG. 4f.1.

FIG. 4g.1 shows a sports strap, in accordance with an embodiment of the present invention, in which pelvis strap 20, leg straps 21, top end 341 of elongation straps 340 and foot straps 22 are fitted with hook 41 and eye 42 fasteners; and bottom end 342 of elongation straps 340 and elongation rings 330 are held together with a stitched seam 45.

FIG. 4g.2 shows a front view of a sports strap, in accordance with an embodiment of the present invention, fitted with the comfort/adjustability related options as shown in FIG. 4g.1.

FIG. 4h.1 shows a sports strap, in accordance with an embodiment of the present invention, in which pelvis strap 20 is fitted with hook 41 and eye 42 fasteners. Leg straps 21 and long leg straps 350 are fitted with slides 44 and hook 41 & eye 42 fasteners.

FIG. 4h.2 shows a front view of a sports strap, in accordance with an embodiment of the present invention, fitted with the comfort/adjustability related options as shown in FIG. 4h.1.

FIG. 4i.1 shows a sports strap, in accordance with an embodiment of the present invention, in which pelvis strap 20, leg straps 21 and foot straps 22 are fitted with press buttons 40.

FIG. 4i.2 is a front view of a sports strap, in accordance with an embodiment of the present invention, fitted with the comfort/adjustability related options as shown in FIG. 4i.1.

FIG. 4j.1 shows a sports strap, in accordance with an embodiment of the present invention, in which pelvis strap 20, leg straps 21 and long leg straps 350 and foot straps 22 are fitted with and hook 41 & eye 42 fasteners.

FIG. 4j.2 shows a front view of a sports strap, in accordance with an embodiment of the present invention, fitted with the comfort/adjustability related options as shown in FIG. 4j.1.

FIGS. 5a.1-5j.1 show possible training variations based on embodiments of the present invention.

FIGS. 5a.1-3 show training variations using embodiments of the present invention.

FIG. 5b shows a training variation using embodiments of the present invention.

FIGS. 5c.1-2 show training variations using embodiments of the present invention.

FIGS. 5d.1-4 show training variations using embodiments of the present invention.

FIGS. 5e.1-2 show training variations using embodiments of the present invention.

FIGS. 5f.1-3 show training variations using embodiments of the present invention.

FIGS. 5g.1-2 show training variations using embodiments of the present invention.

FIG. 5h.1-2 shows training variations using embodiments of the present invention.

FIG. 5i.1 shows a training variation using embodiments of the present invention.

FIG. 5j.1 shows a training variation using embodiments of the present invention.

FIGS. 6a-d show variations of how the foot straps 22 can be worn over the soles of the feet, the heels or over one or more toes.

FIG. 6a is a sectional view of the foot strap as worn over the sole of the foot.

FIG. 6b is a sectional view of two foot straps as worn over the sole of the foot.

FIG. 6c is a sectional view of the foot strap as worn over the heel of the foot.

FIG. 6d is a sectional view of the foot strap as worn over the toes of the foot.

### DETAILED DESCRIPTION OF CERTAIN INVENTIVE EMBODIMENTS

Embodiments of this application relate to a sports strap that helps the user/wearer improve their posture. The device aligns the pelvis horizontally and vertically and strengthens and/or relaxes muscles of the abdomen, the lower back and the lower extremities. The strap may be worn in more than twenty different variations. Depending on the variation, different parts of the body will be affected in different ways. There is a choice of active/activation or relaxed/relaxation mode. Wearing the strap in active motion (including inflexible pelvis strap) makes walking harder by trying to mimic a stair stepping motion, which activates muscles of the lower extremities. It increases the energy expenditure required for walking and helps burn additional calories. Wearing the strap in relaxed motion (only elastic strap, without inflexible pelvis strap) makes the whole body let go of strain, especially the lower part of the body.

The strap may be made out of flexible and inflexible sections and placed at the upper end of the pelvis, focusing on either the front, the back or the side of the pelvis, runs down the legs and ends over the soles of the feet, the heels or over one or more toes. In a preferred embodiment, two rings and an optional horizontal strap ensure that the band stays in position during movement and make the device fit for a wide range of functionality. The design allows the device to be worn under clothing to provide the benefit of training effects while doing other things. Multiple length adjustment options make the strap adaptable to different body geometries and/or different training intensities.

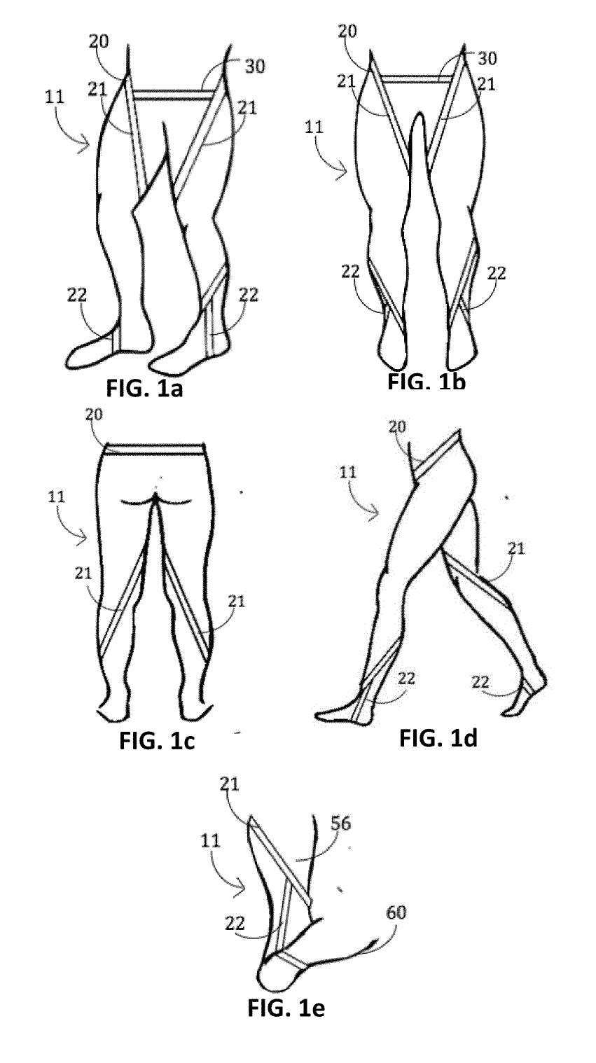

According to an embodiment of the present invention, the sports strap comprises a band, which, in a preferred embodiment, may be the width of a belt, made out of elastic and/or inelastic sections and placed at the user's pelvis, runs down the legs and ends over the soles of the feet, the heels or over one or more toes (see FIGS. 1a-e). In other embodiments, the width may be wider or narrower than a standard belt, depending on application, purpose, resistance requirements, material and other considerations (such as the ability to conceal the strap underneath regular clothing). One of ordinary skill in the art would appreciate that the strap could be constructed at any width, and embodiments of the present invention are contemplated for construction in any appropriate width.

Generally speaking, depending on the position of the pelvis in relation to the position of the legs and the upper body, different muscles of the body are activated or relaxed. In a preferred embodiment, the band comprises an inelastic section configured to run around the pelvis and will therefore influence the position of the pelvis.

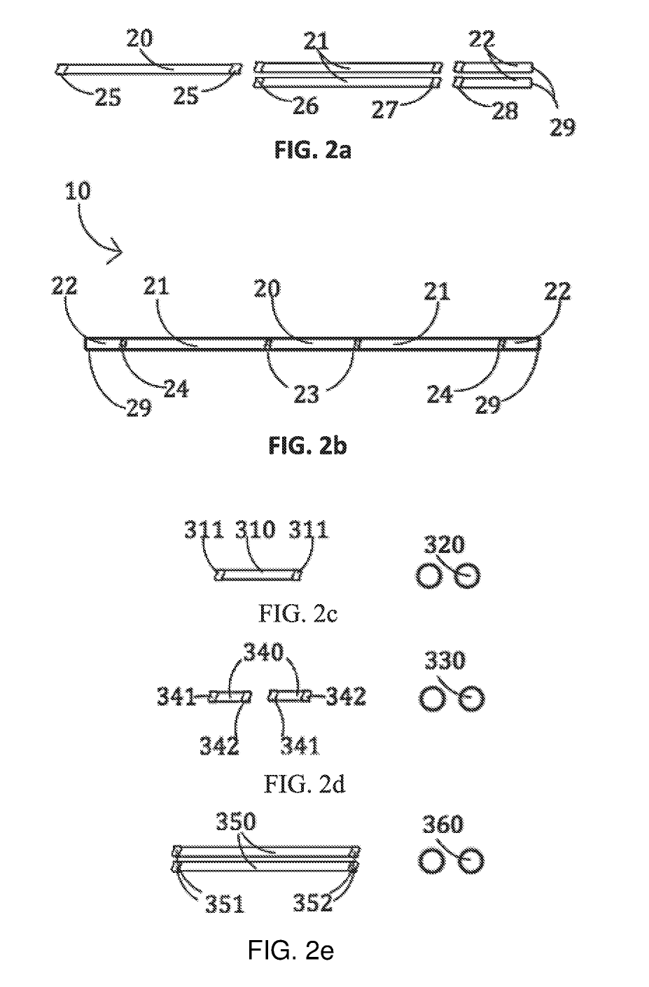

According to a preferred embodiment of the present invention, the sports strap may comprise either one long single band or multiple short single sections (see FIGS. 2a and 2b).

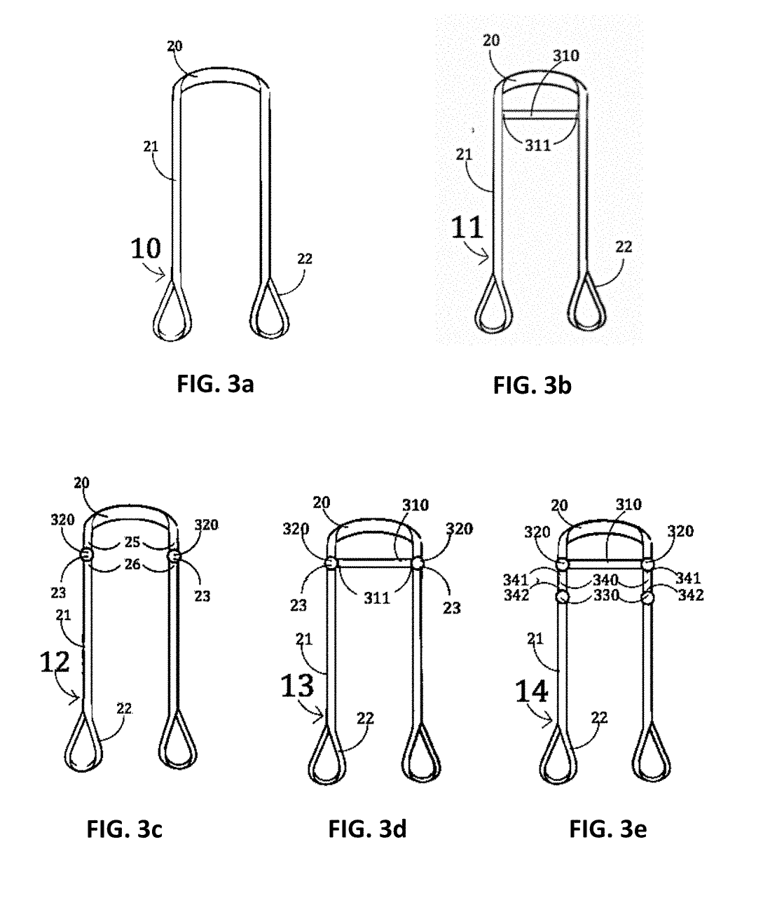

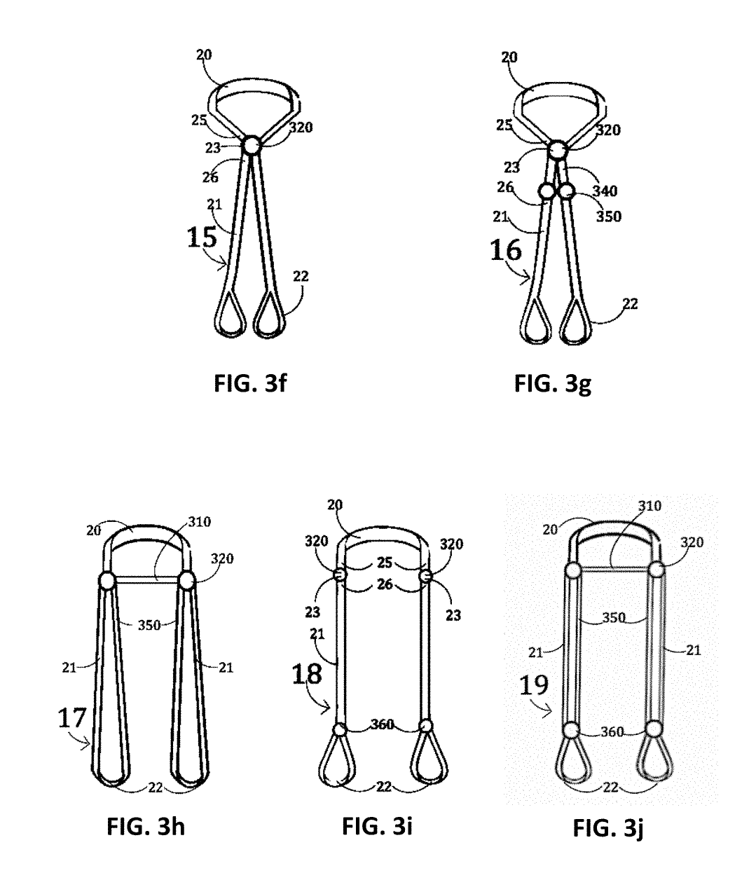

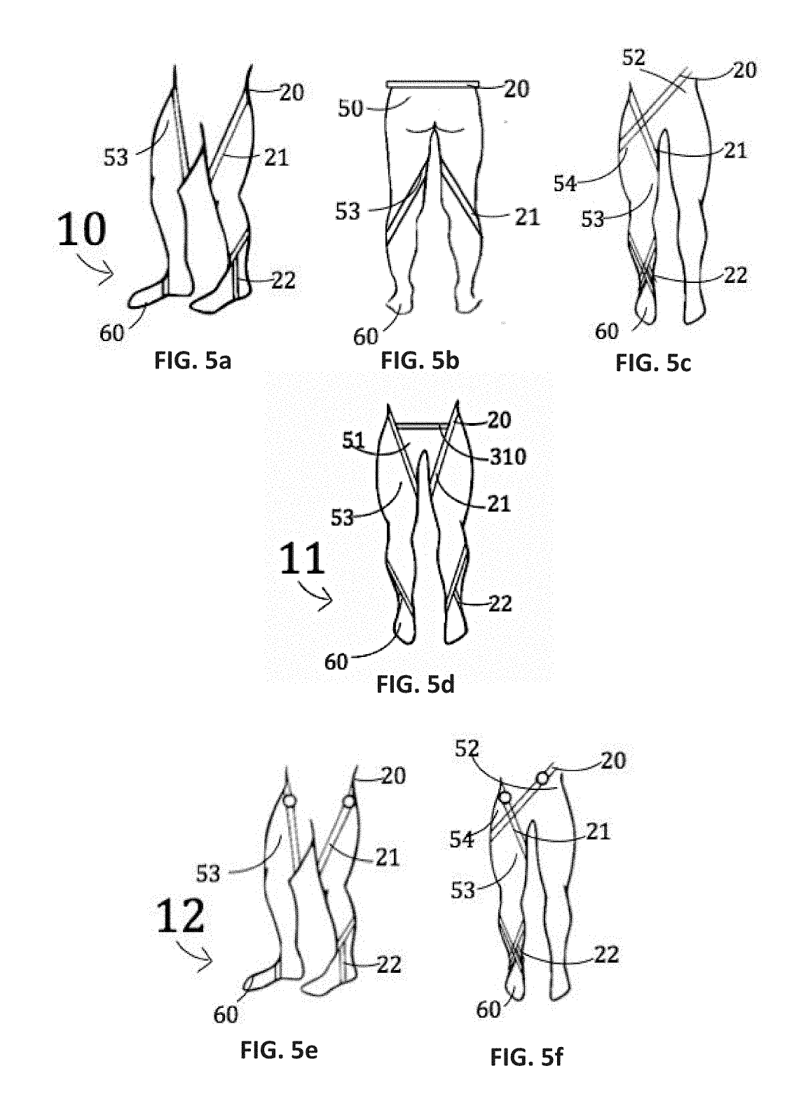

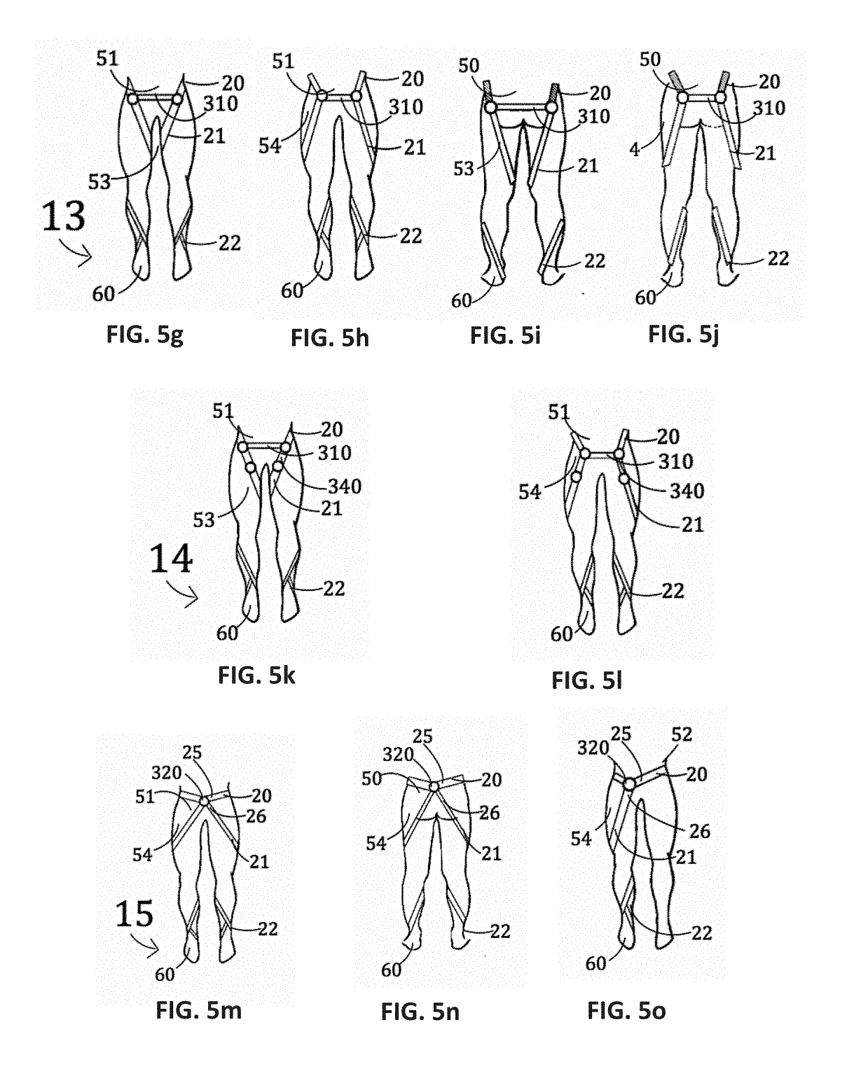

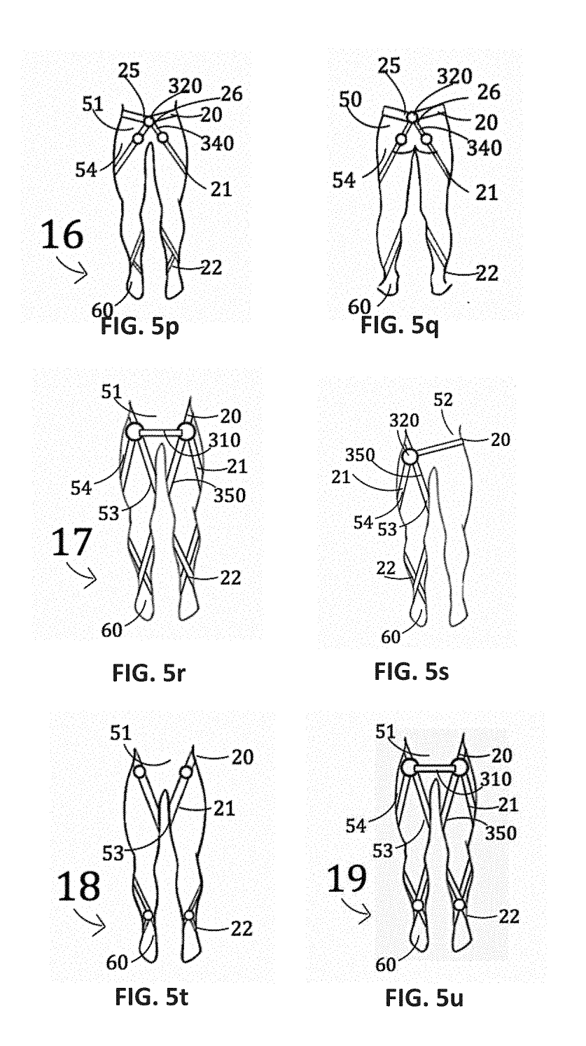

In addition to the “one-band” embodiment (see FIGS. 2a and 2b) optional straps and rings—named as embodiment related options (see FIG. 2c)—may allow different embodiments to be formed (see FIGS. 3a-j), which make the device fit for a wide range of training variations and training effects (see FIGS. 5a-j).

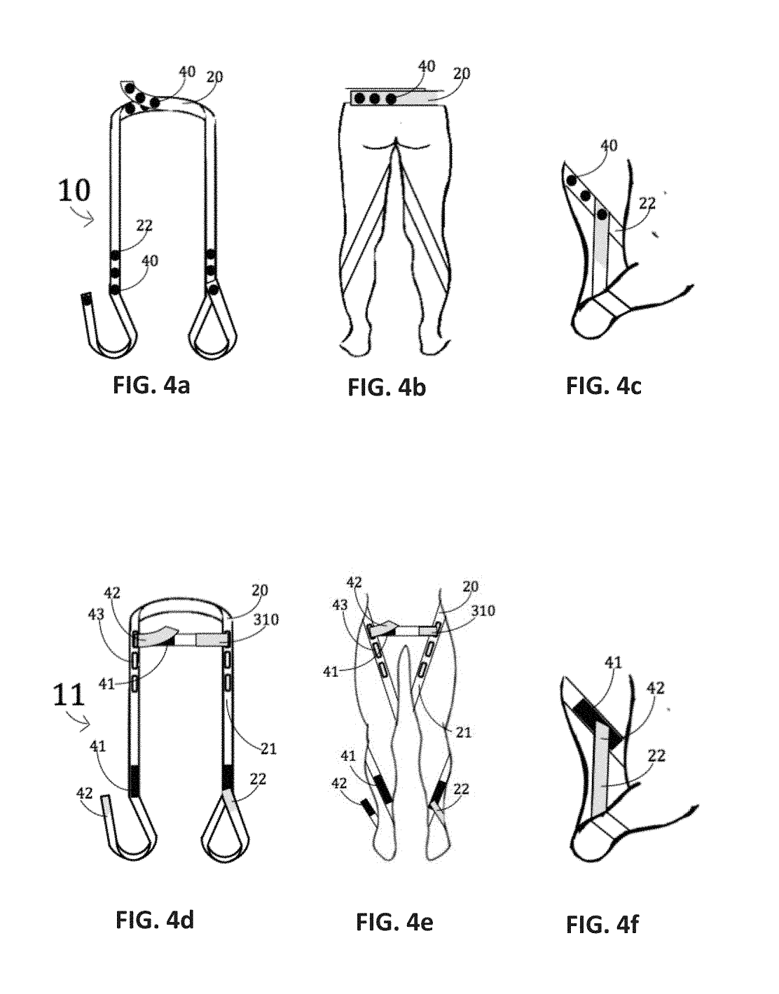

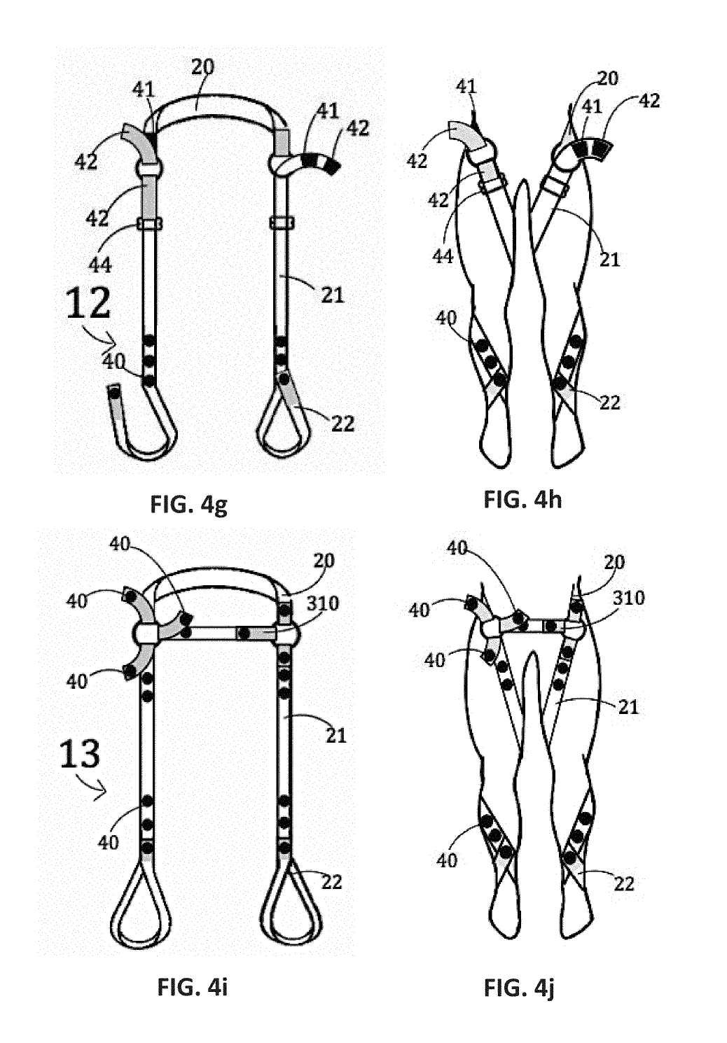

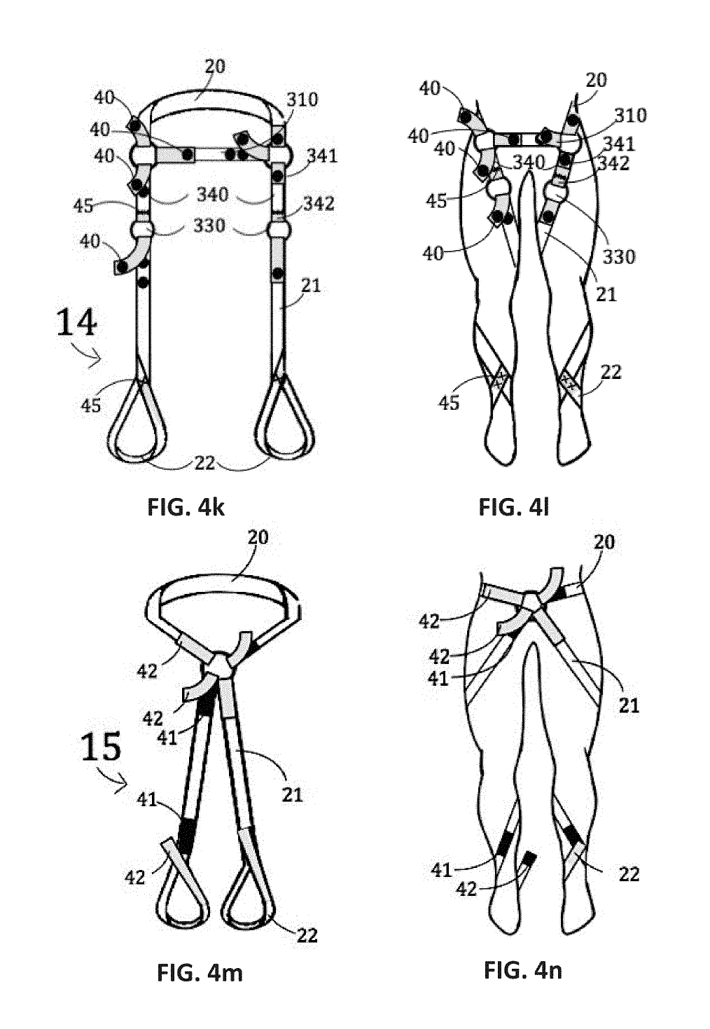

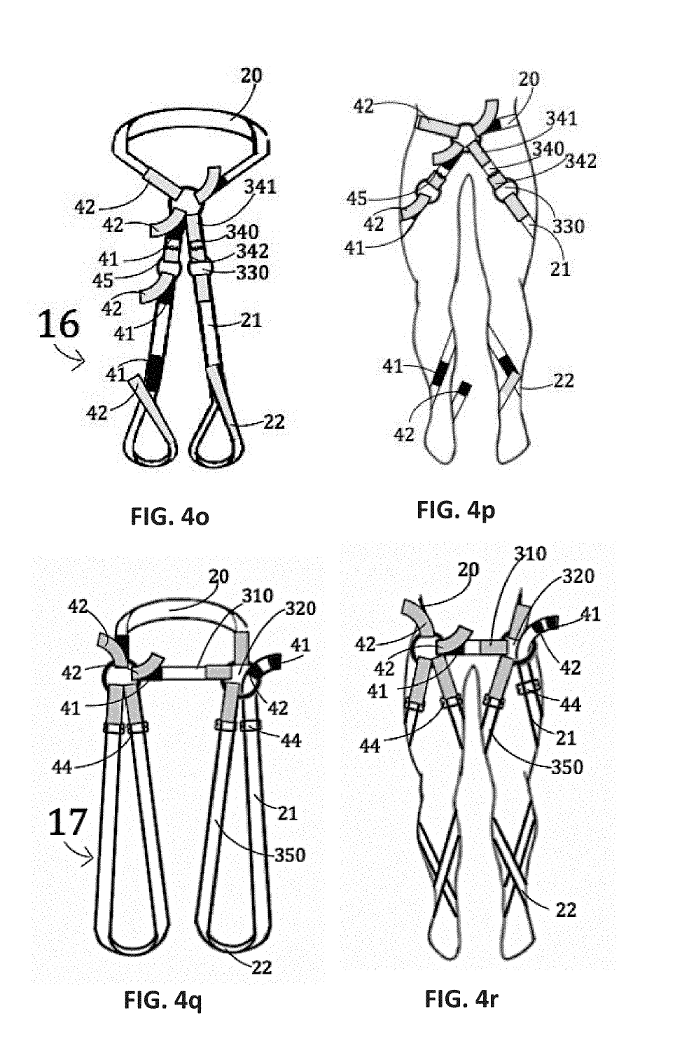

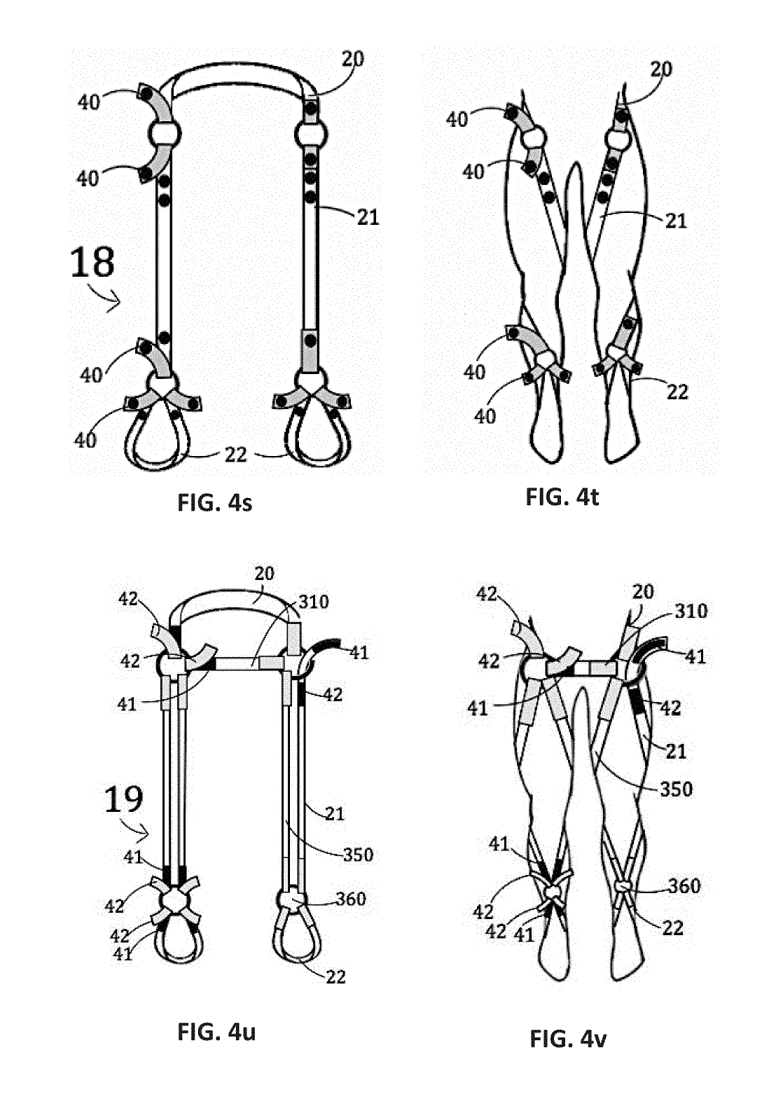

Multiple length adjustment options as well as open/close mechanisms—named as comfort/adjustability related options (see FIGS. 4a-j)—make the strap adaptable to different body geometries and/or different training intensities and at the same time allow a switch from one embodiment to another.

One embodiment of the sports strap can be worn in more than one training variation. Each training variation focuses on special parts of the body and makes special training effects possible (see FIGS. 5a-j).

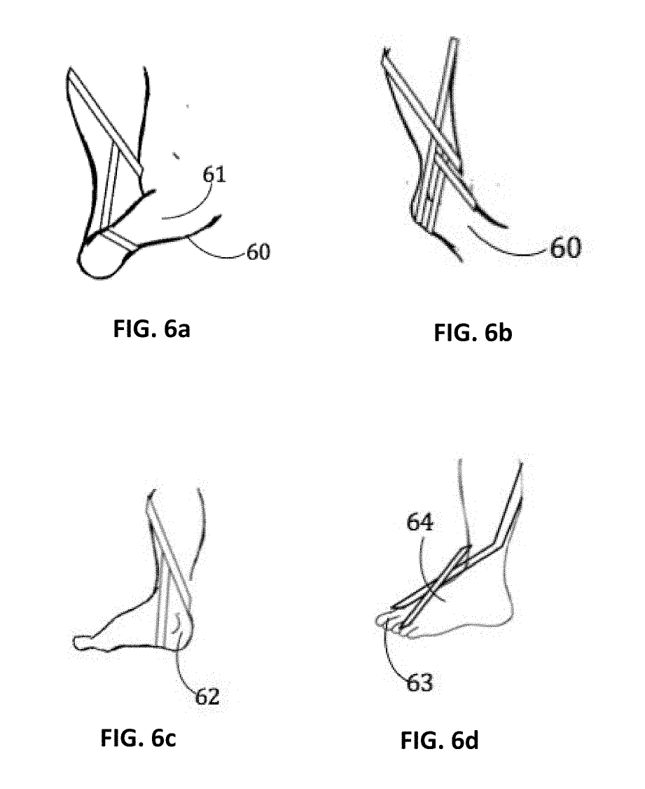

Besides the main training variations (see FIGS. 5a-j), the foot strap may be worn in different ways over the soles of the feet, the heels or over one or more toes (see FIGS. 6a-d).

Turning now to FIGS. 1a, b, 1c and 1d, they show one embodiment 11 of the sports strap. The sports strap is comprised of a pelvis strap 20. The pelvis strap 20 is secured in part by the user's pelvis and by a connected optional center strap 30, which ensures that the pelvis strap 20 stays in place even during various forms of motion by the wearer and influences the training effect. Each end of the pelvis strap 20 run into a single leg strap 21. On its other end, each leg strap runs into a single foot strap 22. The pelvis strap 20 may be comprised of inelastic material. The leg straps may be comprised of elastic material. The foot straps 22 may be comprised of material that is elastic, inelastic or a mix of both.

Turning now to FIG. 1e, an illustration of how the band circles around the foot 60 finally ending at the shin 56 to build up the foot strap 22 is shown. The foot strap 22 is a continuation of leg strap 21. The foot strap 22 may be comprised of material that is elastic, inelastic or a mix of both.

Turning now to FIG. 2a, the separated layout of several straps is shown. One pelvis strap 20 holding on its ends 25 two connections of pelvis strap/leg straps 23. Two leg straps 21 each holding on its top end 26 one connection of pelvis strap/leg strap 23 and on its bottom end 27 one connection of leg straps/foot strap 24. Two foot straps 22 each holding on its top end 28 one connection of leg strap/foot strap 24 and its bottom end 29 will reconnect with the leg strap 21 basically at the position of leg strap/foot strap 24 but may reconnect with the leg strap at any position along the leg strap.

Turning now to FIG. 2b, the layout of several straps (see FIG. 2a) spliced together is shown. It is understood by one having ordinary skill in the art that the straps may be stitched, seamed, glued, stapled, hook and looped together, tied, melted together, held magnetically, or held together via any other shape or form.

Turning now to FIG. 2c, several optional rings & straps, named as embodiment related options, which will allow different embodiments to be formed. One center strap 310, which may be comprised of elastic material, is connected on its ends 311 onto the pelvis strap 20. It ensures that the pelvis strap 20 stays in place even during various forms of motion by the wearer and at the same time will influence the training effect of the sports strap. Two center rings 320 will allow a switch in material between pelvis strap 20, leg straps 21 and center strap 310. Since the pelvis strap 20, the leg straps 21 and the center strap 310 can run along the ring freely, the center ring 320 will support different body geometries and training variations. Two elongation straps 340, which may be comprised of inelastic material, are connected on its top ends 341 onto the center rings 320 and on its bottom end 342 onto to the elongation rings 330. The optional elongation straps 340 and the optional elongation rings 330 will allow an individual setting of the length of the inelastic pelvis strap 20. Medical tests have shown that the longer the inelastic pelvis strap 20 is, the more impact the device has onto the abdominal muscles. On the other hand, if the inelastic pelvis strap 20 reaches too far down towards the knees it can have a negative influence onto the anus muscles. In other words, depending on the length of the inelastic pelvis strap 20, the focus of the training moves from cranial to caudal. Two long leg straps 350 will allow the foot straps 22 to reconnect with the leg straps at any position along the leg straps 21 or even connect with the center ring 320. The bottom end of the long leg strap 352 is connected onto the foot strap 22; the top end of the long leg strap 351 is connected onto the leg strap 21 or onto the center rings 320. Two ankle rings 360 will allow a switch in material between leg straps 21 and foot straps 22 and will allow a simple way to attach/detach two long leg straps 350. Since the leg straps 21 and the foot straps 22 can run along the ring freely, the ankle rings 360 will support different body geometries and training variations.

Turning now to FIGS. 3a-3j, a list of different embodiments is shown. Different embodiments will make the device fit for a wide range of training variations (see FIGS. 5a-j) and training effects.

FIG. 3a shows embodiment 10. Here, the sports strap is constructed of one long single band, regardless if the one band is either constructed of one long single strap or of multiple short single sections spliced together with any seam or alternative form of connection (see FIGS. 2a and 2b). Since it may be advantageous to use the sports strap under everyday clothes throughout the day or during other sports activities, high comport will require less add-ons. Therefore, no additional embodiment related options are attached within embodiment 10.

FIG. 3b shows embodiment 11. Here, the sports strap is constructed of one band (like embodiment 10 FIG. 3a) including the optional center strap 310. The optional center strap 310 ensures that the pelvis strap 20 stays in place even during various forms of motion by the wearer and at the same time will influence the training effect of the sports strap. The optional center strap 310 is connected on its ends 311 onto the pelvis strap 20 or onto the leg straps 21.

FIG. 3c shows embodiment 12. Here, the one band used in embodiment 10 and 11 is parted by two center rings 320 at the meeting point pelvis strap/leg strap 23. The two center rings 320 are connected onto the end of pelvis strap 25 as well as onto the top end of leg strap 26. Two center rings 320 allow a switch in material between pelvis strap 20 and leg straps 21. Since the pelvis strap 20, as well as the leg straps 21, can run along the ring freely, embodiment 12 allows for different training variations and body geometries.

FIG. 3d shows embodiment 13. Here, the one band from embodiment 10 and 11 is parted by two center rings 320 at the meeting point pelvis strap/leg straps 23 (like embodiment 12 FIG. 3c). Additionally, the optional center strap 310 (like embodiment 11 FIG. 3b) is included. The optional center strap 310 is connected on its ends 311 onto the center rings 320.

FIG. 3e shows embodiment 14. Here, the one band from embodiment 10 and 11 is parted by two center rings 320 at the meeting point pelvis strap/leg straps 23 (like embodiment 12 FIG. 3c). Additionally, an optional center strap 310 (like embodiment 11 FIG. 3b) and two inelastic elongation straps 340 as well as two elongation rings 330 are included. The elongation straps 340 are connected on their top ends 341 onto the center rings 320 and their bottom ends 342 onto the elongation rings 330.

FIG. 3f shows embodiment 15. Here, the one band from embodiment 10 and 11 is parted by one center rings 320 at the meeting point pelvis strap/leg straps 23. In general, depending on the position of the meeting point of pelvis strap/leg strap 23 on a horizontal line, the focus of the training moves from lateral to medial. The greatest effect of abdominal training with the sports strap will be gained when the ends 25 of the pelvis strap 20 and the upper ends 26 of the leg straps 21 will join within one point over the center pelvis.

FIG. 3g shows the embodiment 16. Here, the one band from embodiment 10 and 11 is parted by one center rings 320 at the meeting point pelvis strap/leg straps 23 (like embodiment 15 FIG. 3f), including two optional elongation straps 340 and two optional elongation rings 330 (like embodiment 14 FIG. 3e).

FIG. 3h shows the embodiment 17. Here, the sports strap is constructed of a pelvis strap 20, two center rings 320, two leg straps 21, two long leg straps 350 and two foot straps 22. By wearing embodiment 17 the body will require a high level of effort for every step it takes.

FIG. 3i shows embodiment 18. Here, the sports strap is constructed of a pelvis strap 20, two center rings 320, two leg straps 21, two ankle straps 360 and foot straps 22.

FIG. 3j shows embodiment 19. Here, the sports strap is constructed of a pelvis strap 20, two center rings 320, a center strap 310, two leg straps 21, two ankle straps 360 and foot straps 22.

Turning now to FIGS. 4a.1-4j.1, embodiments 10-19 exemplary fitted with comfort/adjustability related, length adjustment, and open/close mechanisms are shown.

FIG. 4a.1 shows embodiment 10 (FIG. 3a) in which pelvis strap 20 is exemplary fitted with press buttons 40 to adjust the length of pelvis strap 20. Foot straps 22 are exemplary fitted with press buttons 40 to adjust the length of foot straps 22 and to open/close foot straps 22. The press buttons 40 may be configured such that a user may adjust the length of the given section of the sports strap based on which buttons are pressed together e.g. beginning buttons, middle buttons, ending buttons.

FIG. 4a.2 shows a backside view of worn embodiment 10 (FIG. 3a) exemplary fitted with comfort/adjustability related options as shown in FIG. 4a.1. In this figure, the length of the pelvis strap 20 is adjusted by using press buttons 40.

FIG. 4a.3 shows foot strap 22 of worn embodiment 10 (FIG. 3a) exemplary fitted with comfort/adjustability related options as shown in FIG. 4a.1. In this figure, the foot strap 22 is secured onto the user's foot using press buttons 40.

FIG. 4b.1 shows embodiment 11 (FIG. 3b) in which pelvis strap 20 is exemplary fitted with buckles 43 to adjust position of center strap 310. Center strap 310 is exemplary fitted with hook 41 & eye 42 fasteners to adjust the length of center strap 310 and/or to open/close/remove center strap 310. Foot straps 22 are exemplary fitted with hook 41 & eye 42 fasteners to adjust the length of foot strap 22 and/or to open/close foot straps 22. It is understood by one having ordinary skill in the art that the hook 41 & eye 42 fasteners may be replaced or supplemented by a hook and loop fastener, or touch fastener. It is also understood that the location of the hook 41 and eye 42 may be exchanged.

FIG. 4b.2 shows a front view of worn embodiment 11 (FIG. 3b) exemplary fitted with comfort/adjustability related options as shown in FIG. 4b.1. In this figure, the center strap 310 is closed on one side but still open on the other side. To close the center strap fully, both ends of the center strap 310 need to use buckles 43 of pelvis strap 20.

FIG. 4b.3 shows foot strap 22 of worn embodiment 11 (FIG. 3b) exemplary fitted with comfort/adjustability related options as shown in FIG. 4b.1. In this figure, the foot strap 22 is secured onto the user's foot using hook 41 and eye 42 fasteners.

FIG. 4c.1 shows embodiment 12 (FIG. 3c) in which pelvis strap 20 is exemplary fitted with hook 41 & eye 42 fasteners to adjust the length of pelvis strap 20 and to switch into embodiment 15 (FIG. 3f) or embodiment 16 (FIG. 3g). Leg straps 21 are fitted with slides 45 and hook 41 & eye 42 fasteners to adjust the length of leg straps 21 and to switch into embodiment 15 (FIG. 3f) or into embodiment 16 (FIG. 3g). Foot straps 22 are exemplary fitted with press buttons 40 to adjust the length of foot straps 22 and to open/close foot straps 22.

FIG. 4c.2 shows a front view of worn embodiment 12 (FIG. 3c) exemplary fitted with comfort/adjustability related options as shown in FIG. 4c.1

FIG. 4d.1 shows embodiment 13 (FIG. 3d) in which pelvis strap 20 is exemplary fitted with press buttons 40 to adjust the length of pelvis strap 20 and to switch into embodiment 15 (FIG. 3f) or into embodiment 16 (FIG. 3g). Center strap 310 is exemplary fitted with press buttons 40 to adjust the length of center strap 310 and to open/close/remove center strap 310. Leg straps 21 are fitted with press buttons 40 to adjust the length of leg straps 21 and/or to switch into embodiment 15 (FIG. 3f) and/or embodiment 16 (FIG. 3g). Foot straps 22 are exemplary fitted with press buttons 40 to adjust the length of foot straps 22 and/or to open/close foot straps 22.

FIG. 4d.2 shows a front view of worn embodiment 13 (FIG. 3d) exemplary fitted with comfort/adjustability related options as shown in FIG. 4d.1.

FIG. 4e.1 shows embodiment 14 (FIG. 3e) in which pelvis strap 20, center strap 310, leg straps 21 and top end 341 of elongation straps 340 are exemplary fitted with press buttons 40 to adjust the length of the straps and to open/close/remove center strap 310 and/or elongation strap 340. Foot straps 22 as well as one bottom end of elongation straps 342 are exemplary fitted with a stitched seam 45. It is understood by one having ordinary skill in the art that the seam 45 may comprise any of the following types: superimposed seam, flat seam, plain seam, lapped seam, abutted seam, French seam, center back seam, inseam, side seam, bound seam, flat seam, decorative/ornamental stitching, edge finishing/neatening, attaching of separate items, or single ply construction. The seamed material may be held together via alternative means such as glue, staples, hook and loop fasteners, hook and eye fasteners, alternative fasteners, ties, melted together, magnetism, or held together in any other shape or form. Various other seams may alternatively be located on any portion of the sports strap.

FIG. 4e.2 shows a front view of worn embodiment 14 (FIG. 3e) exemplary fitted with comfort/adjustability related options as shown in FIG. 4e.1

FIG. 4f.1 shows embodiment 15 (FIG. 3f) in which pelvis strap 20, leg straps 21 and foot straps 22 are exemplary fitted with hook 41 and eye 42 fasteners and center ring 320 to adjust the length of pelvis strap 20, leg straps 21, foot straps 22, and to switch into other embodiments.

FIG. 4f 2 shows a front view of worn embodiment 15 (FIG. 3f) exemplary fitted with comfort/adjustability related options as shown in FIG. 4f.1

FIG. 4g.1 shows embodiment 16 (FIG. 3g) in which pelvis strap 20, leg straps 21, top end 341 of elongation straps 340 and foot straps 22 are exemplary fitted with hook 41 and eye 42 fasteners to adjust the length of pelvis strap 20, leg straps 21, elongations straps 340 and foot straps 22, and to switch into other embodiments. Bottom end 342 of elongation straps 340 and elongation rings 330 are exemplary held together with a stitched seam 45.

FIG. 4g.2 shows a front view of worn embodiment 16 (FIG. 3g) exemplary fitted with comfort/adjustability related options as shown in FIG. 4g.1

FIG. 4h.1 shows embodiment 17 (FIG. 3h) in which pelvis strap 20 is exemplary fitted with hook 41 and eye 42 fasteners. Leg straps 21 and long leg straps 350 are exemplary fitted with slides 44 as well as hook 41 & eye 42 fasteners. Pelvis strap 20, center strap 310, leg straps 21 and long leg straps 350 are connected to center rings 320. Leg straps 21, long leg straps 350 and foot strap 22 are exemplary constructed of one long band.

FIG. 4h.2 shows a front view of worn embodiment 17 (FIG. 3h) exemplary fitted with the comfort/adjustability related options as shown in FIG. 4h.1.

FIG. 4i.1 shows embodiment 18 (FIG. 3i), in which pelvis strap 20, leg straps 21 and foot straps 22 are fitted with press buttons 40 to adjust the length of pelvis strap 20, leg straps 21 and foot straps 22 and to switch into other embodiment.

FIG. 4i.2 is a front view of a sports strap, in accordance with an embodiment of the present invention, fitted with the comfort/adjustability related options as shown in FIG. 4i.1.

FIG. 4j.1 shows embodiment 19 (FIG. 3j), in which pelvis strap 20, center strap 310, leg straps 21, long leg straps 350 and foot straps 22 are fitted hook 41 & eye 42 fasteners to adjust the length of pelvis strap 20, center strap 310, leg straps 21 long leg straps 350 and foot straps 22 and to switch into other embodiment.

FIG. 4i.2 is a front view of a sports strap, in accordance with an embodiment of the present invention, fitted with the comfort/adjustability related options as shown in FIG. 4j.1.

Turning now to FIGS. 5a.1-5j.1 showing possible training variations based on embodiments 10-19 (FIG. 3a-3j)

FIG. 5a.1 shows embodiment 10 worn with pelvis strap 20 over the backside of the pelvis 50. Leg straps 21 worn on the inner thighs 53. Foot straps 22 worn over feet 60. This variation supports a horizontal and vertical balancing of the pelvis and strengthens the muscles of the lower extremities. FIG. 5a.1 is a front oblique view.

FIG. 5a.2 shows embodiment 10 worn with pelvis strap 20 over the backside of the pelvis. FIG. 5a.2 is a back view of FIG. 5a.1.

FIG. 5a.3 shows the embodiment 10 worn with pelvis strap 20 centered over one hip 52 (ilium bone). One leg strap worn on the outer thigh 54, one leg strap on the inner thigh 53. Both foot straps 22 are ending over one foot 60. This variation triggers the pelvis to lean to one side and strengthens the muscles of one leg and one foot and gives space to the hip joint of the other side.

FIG. 5b shows the embodiment 11 worn with pelvis strap 20 over the backside of the pelvis 50. Center strap 310 worn over front side of pelvis 51. Leg straps 21 worn on the inner thighs 53. Foot straps 22 worn over feet 60. This variation supports a horizontal and vertical balancing of the pelvis and strengthens the muscles of the lower extremities.

FIG. 5c.1 shows the embodiment 12 worn with pelvis strap 20 over the backside of the pelvis 50. Leg straps 21 worn on the inner thighs 53. Foot straps 22 worn over feet 60. This variation supports a horizontal and vertical balancing of the pelvis and strengthens the muscles of the lower extremities.

FIG. 5c.2 shows embodiment 12 worn with pelvis strap 20 centered over one hip 52 (ilium bone). One leg strap worn on the outer thigh 54, one leg strap on the inner thigh 53. Both foot straps 22 are ending over one foot 60. This variation triggers the pelvis to lean to one side and strengthens the muscles of one leg and one foot and gives space to the hip joint of the other side.

FIG. 5d.1 shows the embodiment 13 worn with pelvis strap 20 over the backside of the pelvis 50. Center strap 310 worn over front side of pelvis 51. Leg straps 21 worn on inner thighs 53. Foot straps 22 worn over feet 60. This variation supports a horizontal and vertical balancing of the pelvis and strengthens the muscles of the lower extremities and triggers the thighs to rotate outwards.

FIG. 5d.2 shows the embodiment 13 worn with pelvis strap 20 over the backside of the pelvis 50. Center strap 30 worn over front side of pelvis 51. Leg straps 21 worn on outer thighs 54. Foot straps 22 worn over feet 60. This variation supports a horizontal and vertical balancing of the pelvis, strengthens the muscles of the lower extremities and triggers the thighs to rotate inwards.

FIG. 5d.3 shows the embodiment 13 worn with pelvis strap 20 over the front side of the pelvis 51 in the height of the navel. Center strap 310 worn over backside of pelvis 50. Leg straps 21 inside of the thighs 53. Foot straps 22 worn over feet 60. This variation supports an horizontal and vertical balancing of the pelvis, strengthens the muscles of the lower extremities, reduces the length between the navel and the pubic bone which animates the user to focus on the abdominal muscles and triggers the thighs to rotate outwards.

FIG. 5d.4 shows the embodiment 13 worn with pelvis strap 20 over the front side of the pelvis 51 in the height of the navel. Center strap 310 worn over backside of pelvis 50. Leg straps 21 outside of the thighs 54. Foot straps 22 worn over feet 60. This variation supports an horizontal and vertical balancing of the pelvis, strengthens the muscles of the lower extremities, reduces the length between the navel and the pubic bone which animates the user to focus on the abdominal muscles and triggers the thighs to rotate inwards.

FIG. 5e.1 shows the embodiment 14 worn with pelvis strap 20 over the backside of the pelvis 50. Center strap 310 worn over front side of pelvis 51. Leg straps 21 and elongation straps 340 inside of the thighs 53. The inelastic pelvis strap 20 together with the inelastic elongation strap 340 will provide a strong push forward onto the pelvis and will therefore activate the abdominal muscles in a very strong way. This variation supports also triggers the thighs to rotate outwards.

FIG. 5e.2 shows the embodiment 14 worn with pelvis strap 20 over the backside of the pelvis 50. Center strap 310 worn over front side of pelvis 51. Leg straps 21 and elongation straps 340 outside of the thighs 54. Foot straps 22 worn over feet 60. The inelastic pelvis strap 20 together with the inelastic elongation strap 340 will provide a strong push forward onto the pelvis and will therefore activate the abdominal muscles in a very strong way. This variation supports also triggers the thighs to rotate inwards.

FIG. 5f.1 shows the embodiment 15 worn with pelvis strap 20 over the backside of the pelvis 50. One center ring 320 worn over front side of pelvis 51. Leg straps 21 outside of the thighs 54. Foot straps 22 worn over feet 60. This variation supports a horizontal and vertical balancing of the pelvis, strengthens the muscles of the lower extremities and triggers the thighs to rotate inwards. A great effect of abdominal training will be gained because the ends 25 of the pelvis strap 20 and the top ends 26 of the leg straps 21 will join within one point over the center of the pelvis.

FIG. 5f.2 shows the embodiment 15 worn with pelvis strap 20 over the front side of the pelvis 51. One center ring 320 worn over backside of pelvis 50. Leg straps 21 worn on the outer thighs 54. Foot straps 22 worn over feet 60. A great effect of abdominal training will be gained because the ends 25 of the pelvis strap 20 and the top ends 26 of the leg straps 21 will join within one point over the center pelvis. At the same time, the length between the navel and the pubic bone will be reduced, thus producing in the user a focus on the abdominal muscles.

FIG. 5f.3 shows the embodiment 15 worn with pelvis strap 20 centered over one hip 52 (ilium bone) using just one leg strap 21 over a single leg. One leg strap 21 worn on the outer thigh 54. Single foot strap 22 worn over a single foot 60. This variation triggers the pelvis to lean to one side and strengthens the muscles of one leg and one foot and gives space to the hip joint of the other side.

FIG. 5g.1 shows the embodiment 16 worn with pelvis strap 20 over the backside of the pelvis 50. One center ring 320 worn over front side of pelvis 51. Leg straps 21 and elongation straps 340 worn over outer thighs 54. Foot straps 22 worn over feet 60. The inelastic pelvis strap 20 together with the inelastic elongation strap 340 will provide a strong push forward onto the pelvis and because the ends 25 of the pelvis strap 20 and the top ends 26 of the leg straps 21 will join within one point over the center pelvis. This variation may provide a very strong effect on the abdominal muscles.

FIG. 5g.2 shows the embodiment 16 worn with pelvis strap 20 over the front side of the pelvis 51. One center ring 320 worn over backside of pelvis 50. Leg straps 21 and elongation straps 33 worn over outer thighs 54. Foot straps 22 worn over feet 60. The inelastic pelvis strap 20 together with the inelastic elongation strap 340 will provide a strong push forward onto the pelvis. And, because the ends 25 of the pelvis strap 20 and the top ends 26 of the leg straps 21 will join within one point over the center pelvis, this variation will provide a very strong effect on the abdominal muscles. At the same time, the length between the navel and the pubic bone will be reduced, thus producing in the user a focus on the abdominal muscles.

FIG. 5h.1 shows embodiment 17 worn with pelvis strap 20 over the backside of the pelvis 50. Leg straps 21 worn over outer thighs 54 and long leg straps 350 worn over inner thighs 53. Foot straps 22 worn over feet 60. By wearing embodiment 17 the body will require a high level of effort for every step to take. Embodiment 17 provides the most powerful trainings variation for the lower extremities.

FIG. 5h.2 shows the embodiment 17 worn with pelvis strap 20 centered over one hip 52 (ilium bone) using one center ring 320. Leg strap 21 worn on the outer thigh 54 and long leg strap 350 worn on the inner thigh 53. Single foot strap 22 worn over a single foot 60. This variation triggers the pelvis to lean to one side and strengthens the muscles of one leg and one foot and gives space to the hip joint of the other side.

FIG. 5i.1 shows a training variation using embodiments of the present invention.

FIG. 5j.1 shows a training variation using embodiments of the present invention.

Turning now to FIGS. 6a-d variations of how the foot straps 22 can be worn over the soles of the feet, the heels or over one or more toes are shown. These variations may be used with any of the embodiments of the invention disclosed herein.

FIG. 6a shows foot strap 22 worn over the sole 61 of the foot. This variation may allow for the tension of the foot strap to be exerted on the sole 61 of the user's foot 56

FIG. 6b shows two foot straps 22 worn over the sole 61 of the foot. This variation may allow for additional focus/tension to be placed on a single foot of the user.

FIG. 6c shows foot strap 22 worn over the heel 62 of the foot. This variation will trigger to plant the foot on the outside edge of the foot, which will give space to the joint of the big toe.

FIG. 6d shows foot straps 22 worn over the toes 63 of the foot and the top of the foot 64. This variation may allow for strength training, or correction of the user's toes 63.

It should be understood that the aspects, features and advantages made apparent from the foregoing are efficiently attained and, since certain changes may be made in the disclosed inventive embodiments without departing from the spirit and scope of the invention, it is intended that all matter contained herein shall be interpreted as illustrative and not in a limiting sense.

In still further embodiments of the present invention, various configurations of the sport strap detailed herein may be integrated into articles of clothing or other apparel items. For instance, a sports strap may be integrated into a pair of workout pants, with the various components of the sports strap formed inside seams or other areas of the pants. Other exemplary articles of clothing or apparel may include, but are not limited to, yoga pants, capris, jeans, dress pants, under garments (e.g., long johns), compression sportswear, or any combination thereof. One of ordinary skill in the art would appreciate that there are numerous types of articles of clothing or other apparel that the sports strap may be integrated of formed with, and various methods for incorporating of forming the sports strap with such articles of clothing of other apparel, and embodiments of the present invention are contemplated for use with any such article of clothing or other apparel and methods of integrating or incorporating the sports strap therein.

All of the features specified in the description of the figures, in the claims and in the introductory part of the description may be used both individually and also in any desired combination with one another. The invention is therefore not restricted to the described or claimed combinations of features. In fact, all combinations of features should be regarded as being disclosed.

It will be seen that the objects set forth above, among those made apparent from the preceding description, are efficiently attained, and since certain changes may be made without departing from the spirit and scope of the invention, it is intended that all matter contained in the above description or shown in the accompanying drawing shall be interpreted as illustrative and not in a limiting sense.

It is also to be understood that the following claims are intended to cover all of the generic and specific features of the invention herein disclosed and all statements of the scope of the invention that, as a matter of language, might be said to fall there between.

What is claimed is:

1. A sports strap for improving body posture and back and abdominal muscles and the muscles of the lower extremities, said strap comprising:
a band with a pelvis strap forming a center portion of said band, first and a second side part, said first side part forming a first leg strap and said second part forming a second leg strap,
wherein each of said first and second leg strap is connected a distal point from the pelvis strap, said distal point connection forming a first foot strap loop on said first leg strap and a second foot strap loop on said second leg strap.

2. The strap of claim 1, wherein the assembly of the pelvis strap, first and second leg straps and first and second foot straps, comprises a plurality of segments, wherein each segment has a variable length.

3. The strap of claim 1, wherein the band is a single, firmly connected band and wherein the connections are not adjustable.

4. The strap of claim 1, wherein the assembly of the pelvis strap, first and second leg straps and first and second foot straps are connected with one or more connectors to allow adjustment of one or more of the pelvis strap, first and second leg straps and first and second foot straps.

5. The strap of claim 4, wherein said one or more connectors are selected from the group consisting of buttons, hooks and hook and loop fasteners, glides, slides, strap connectors, closures, rings, links, buckles, extra-straps, seam.

6. The strap of claim 1, wherein the assembly of the pelvis strap, first and second leg straps and first and second foot straps are connected with one or more connectors to allow for a plurality of configurations whilst allowing for adjustment of the pelvis strap, first and second leg straps and first and second foot straps.

7. The strap of claim 1, wherein the pelvis strap is inelastic and at least a segment of one or more of said first and second leg strap is elastic and each of the first and second foot straps is inelastic.

8. The strap of claim 1, wherein the pelvis strap is inelastic and at least a segment of one or more of said first and second leg strap is elastic and each of the first and second foot straps is elastic.

9. A sport strap for improving body posture, said strap comprising:
a pelvis strap connected to two leg straps;
each of said leg straps terminating in a foot strap, wherein the foot strap is formed into a loop such that a foot of a user may be retained within said loop,
wherein said pelvis strap is configured to be looped around the pelvis of an individual and each of said two leg straps is configured to wrap around a single leg of the user and retained upon the pelvis and legs of said user via tension provided by extension of the sport strap from the pelvis down to the feet of the user.

10. The strap of claim 9 wherein the pelvis strap further comprises a soft padding on an inner side to provide for wearing comfort.

11. The strap of claim 9 further comprising a horizontal center strap, with an open-close mechanism allowing for adjustment, wherein said horizontal center strap is configured to connect to one or more of the one or more fasteners.

12. The strap of claim 9 further comprising a length adjustment mechanism configured to make the pelvis strap adaptable to different size and training requirements.

13. The strap of claim 9 further comprising a buckle configured to attach the pelvis strap and the leg strap.

14. The strap of claim 9 further comprising a leg strap adjustment mechanism configured to allow for length adjustment based on the user's size or the resistance of the leg band.

15. The strap of claim 9 further comprising a buckle configured to attach the leg straps and the foot straps.

16. The strap of claim 9 further comprising a foot strap adjustment mechanism to allow for length adjustment of the foot strap based on the user's size of the feet.
